(19) United States
(12) Patent Application Publication
(10) Pub. No.: US 2014/0257254 A1
(43) Pub. Date: Sep. 11, 2014

(54) Fractional Handpiece for Dermatological Treatments

(71) Applicant: Biolase, Inc., Irvine, CA (US)

(72) Inventors: Dmitri Boutoussov, Dana Point, CA (US); Vladimir Netchitailo, Livermore, CA (US); Maziar Salehi, Irvine, CA (US); Julio Cely, Mission Viejo, CA (US); Vladimir Lemberg, Santa Clara, CA (US)

(21) Appl. No.: 14/204,479

(22) Filed: Mar. 11, 2014

Related U.S. Application Data
(60) Provisional application No. 61/776,327, filed on Mar. 11, 2013.

(51) Int. Cl. A61N 5/06; A61B 18/20

(52) U.S. Cl. CPC A61N 5/0616; A61B 18/203
USPC 606/3; 606/9

(57) ABSTRACT

A medical device includes a housing that is moved along a surface of a target tissue in a longitudinal direction. One or more supply lines conduct air and water to the housing. A pulse emitter emits electromagnetic pulses toward the surface at a repetition rate for the pulses to produce ablation holes in the tissue. The pulse emitter includes optical components and is configured to direct the air against the optical components to keep the optical components clean. One or more nozzles emit the water and the air in an air/water spray to moisturize and cool the target tissue prior to laser application.

### CROSS-REFERENCE TO RELATED APPLICATION

This claims priority to U.S. Provisional Application No. 61/776,327, filed Mar. 11, 2013, hereby incorporated herein by reference.

### TECHNICAL FIELD

This relates to a dermatological laser treatment device used for fractional laser treatment of skin.

### BACKGROUND

A fractional laser treatment device is used to treat skin conditions. A medical practitioner holds the device against a patient's skin. The device emits, toward the skin, laser pulses that produce a matrix of small ablation areas on the skin. The ablation areas are later filled in by growth from surrounding unablated skin tissue, which results in improved skin texture.

### SUMMARY

A medical device includes a housing that is moved along a surface of a target tissue in a longitudinal direction. One or more supply lines conduct air and water to the housing. A pulse emitter emits electromagnetic pulses toward the surface at a repetition rate for the pulses to produce ablation holes in the tissue. The pulse emitter includes optical components and is configured to direct the air against the optical components to keep the optical components clean. One or more nozzles emit the water and the air in an air/water spray to moisturize and cool the target tissue prior to laser application.

A medical device includes a housing configured to be moved along a surface of a target tissue in a longitudinal direction. A movement sensor measures a movement parameter, which might be longitudinal speed or displacement, of the housing relative to the surface. A pulse emitter emits electromagnetic pulses toward the surface at a repetition rate, for the pulses to produce ablation holes in the tissue. A controller controls the repetition rate, based on the measured movement parameter, so that the ablation holes are spaced apart along the longitudinal direction.

In one example, the pulses are laser pulses. The movement parameter is longitudinal speed, the sensor outputs a speed signal indicative of the longitudinal speed, and the controller controls the repetition rate to be a function of the longitudinal speed indicated by the speed signal. Alternatively, the movement parameter is longitudinal displacement, the sensor is configured to output a displacement signal indicative of the longitudinal displacement, and the controller is configured to control emission of the pulses to be a function of the displacement signal.

In one example, the sensor includes a roller that is rotatably secured to the housing and configured to roll against the surface as the housing is moved longitudinally along the surface, so that rate of rotation of the roller is proportional to speed of the longitudinal movement. The sensor is configured measure an angular movement parameter, which may be angular speed or angular displacement, of the roller. The angular movement parameter may be angular speed of the roller, and the controller may control the repetition rate to be a function of the angular speed. Alternatively, the angular movement parameter is angular displacement of the of the roller, and the controller controls the laser emitter to space apart the pulses as a function of the angular displacement.

In one example, the roller is a first roller at a first side of the housing and the angular movement parameter is a first angular movement parameter. The movement sensor includes a second roller at a laterally opposite second side of the housing, that is rotatably secured to the housing to rotate independently of rotation of the first roller and to roll against the surface. The movement sensor measures a second angular movement parameter, comprising angular speed or angular displacement, of the second roller.

### BRIEF DESCRIPTION OF THE FIGURES

FIG. 1 is a schematic diagram of an example medical laser system, including a laser handpiece, for performing fractional laser treatment procedures.

FIG. 2 is a perspective view of the handpiece and ablation holes generated by the handpiece.

### DETAILED DESCRIPTION

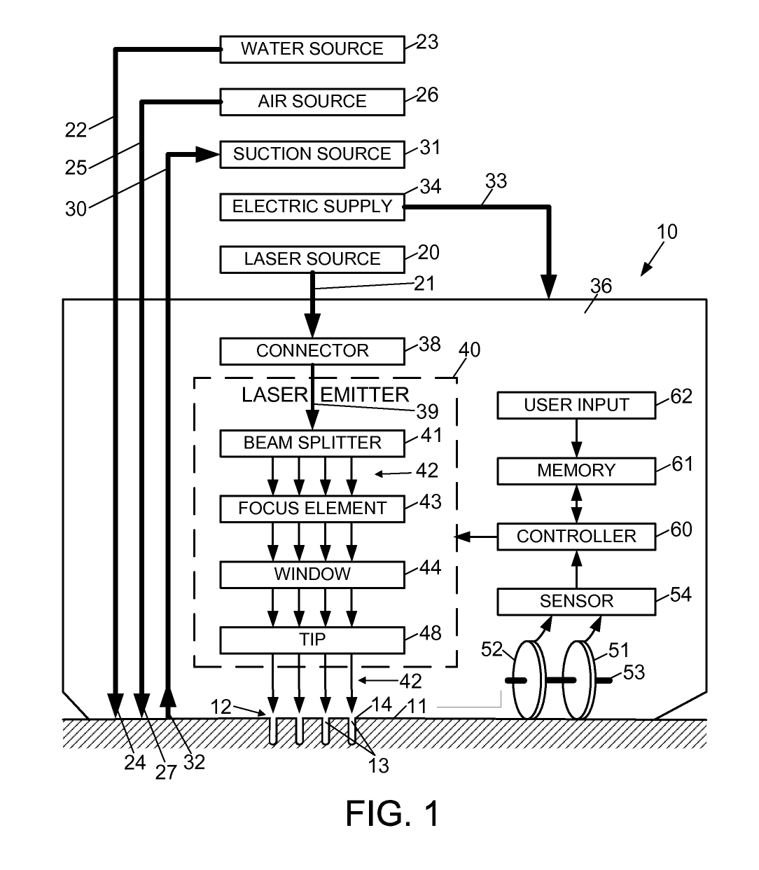

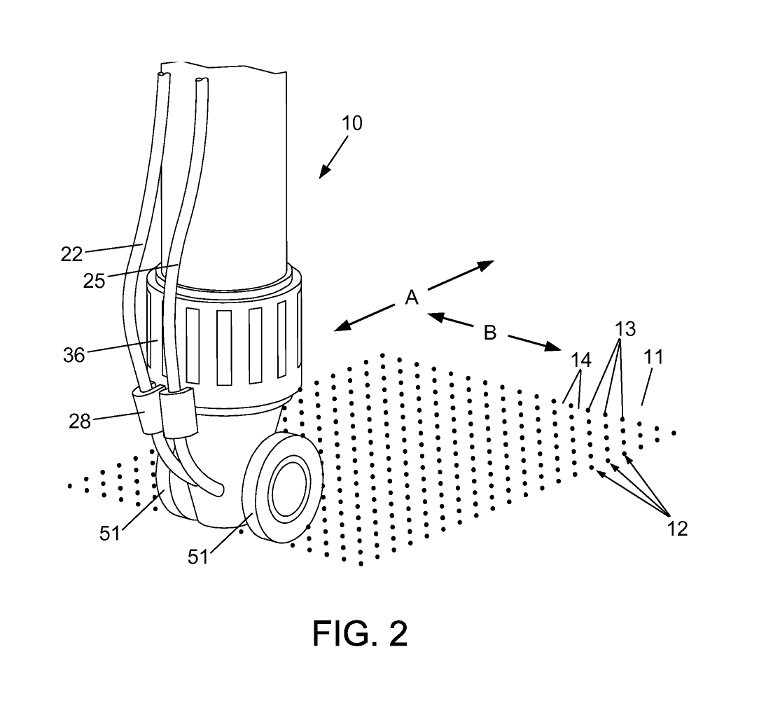

FIG. 1 is a block diagram of a medical system for electromagnetic beam treatment of biological tissue. The system includes a handpiece 10, a perspective view of which is shown in FIG. 2. Referring to FIGS. 1-2, a medical practitioner (user) holds the handpiece 10 against a surface 11 of the target tissue to be treated. As the user moves the handpiece 10 along the surface 11, the handpiece 10 emits electromagnetic pulses (short-duration electromagnetic energy beams) toward the tissue surface. The tissue is ablated in a fractional manner, in that the pulses are timed and oriented so as to ablate an array of small areas 13 in the tissue. The handpiece 10 tracks its progress in traversing the surface 11 in order to time the pulses to be properly spaced apart along the surface 11. Unablated tissue 14 surrounding each ablated area will then quickly rejuvenate the small ablated areas 13.

In this example, the target tissue is skin, the treatment is a dermatological treatment, the electromagnetic pulse is a laser pulse, and the handpiece is a fractional laser handpiece. The ablated area 13 may be termed as an ablation, perforation, spot, pixel, dot, hole, pit or crater. Each perforation 13 stimulates growth of new collagen. The new collagen improves the skin's texture and tone to smooth fine lines and wrinkles Unablated skin 14 surrounding each perforation remains intact, which allows the skin's top layer 11 to heal rapidly from the edge 14 of each perforation 13.

In the following description of the system's hardware, the handpiece 10 is configured to be moved along the surface 11 in a “longitudinal” direction (arrow “A” in FIG. 2), which is perpendicular to a “lateral” direction along the surface 11 (arrow “B” in FIG. 2).

The example system includes an ablation energy source 20 that generates the electromagnetic energy for ablating the tissue 11. In this example, the source 20 is a laser source that generates laser energy. Example laser sources are a Er,Cr:YSGG laser, a Er:YAG laser, a diode laser, a Nd:YAG laser, an Argon laser module or CO2 laser module. Wavelength in this example is in the range of 1.9-3.0 micrometers, and may be in the rage 2.7-2. 85 micrometers range, as this provides a deeper penetration than Er:YAG. Energy density within each spot should be above the ablation threshold of the tissue being treated. The energy density to be used for each type of treatment may be determined based on a balance of pulse energy and number of spots and spot size.

In this example, the laser source 20 is outside the handpiece 10. It may alternatively be within, and part of, the handpiece 10.

An ablation energy supply line 21, which may include one or more optical fibers, conducts the electromagnetic energy, in this example laser energy, from the ablation energy source 20 to the handpiece 10. A water supply line 22 conducts water from a water source 23 to a water nozzle 24 at the front (laser emitting end) of the handpiece 10. An air supply line 25 conducts air from an air source 26 (air pump) to an air nozzle 27 at the front of the handpiece 10. A suction line 30 provides suction from a suction source 31 to a suction port 32 at the front of the handpiece 10. An electrical supply line 33 provides electrical power from a power supply 34 to circuitry (e.g., controller) of the handpiece 10.

The nozzles 24, 27 emit and direct the water and air to the skin surface 11. The nozzles emit the water and the air in the form of an air/water spray that moisturizes and cools the tissue prior to laser application. The air and water, along with the suction, keep the treatment area 11 cool and free from debris. The water may be sprayed on the treatment area 11 in a fine high-pressure mist to cool and wet the treatment area, react with the laser pulse to achieve the ablation, and stimulate ablation of superficial less-hydrated skin. The air and the suction may also be channeled into the handpiece 10 to keep optical components of the handpiece 10 clean.

The handpiece 10 includes a housing 36 configured to be gripped by the user and moved along the skin surface 11. The housing 36 is ergonomically designed to facilitate holding and operating of the handpiece 10.

A connector 38 at a rear end of the housing 36 is removably coupled to the laser energy supply line 21 to receive the laser energy. Optical fibers 39 within the housing 36 channel the laser energy from the connector 38 to a laser pulse emitter 40 within the housing 36.

The pulse emitter 40 in this example includes the following components: A beam splitter 41, such as an optical assembly with a diffractive optical element, splits the laser energy 39 into a laterally extending row (series) of separate laser pulses 42 that are laterally spaced apart. The laser pulses are emitted toward the surface 11 so as to produce a laterally extending row 12 of ablation holes. The laser pulses for each row 42 may be emitted simultaneously or sequentially. The lateral spacing distance between holes is user selectable. A focusing element 43 includes one or more lens elements for focusing the laser pulses. A protective window 44 protects the optical components from damage or debris. A laser tip 48 is secured to the end of the emitter 40. The tip 48 may contact the skin surface 11 when applying the laser pulses to the skin. The tip 48 may be disposable, so that a different sanitary tip may be used for each patient. The emitter 40 includes optical components and is configured to direct the air against the optical components to keep the optical components clean.

Support rollers 51, 52 (wheels) of the handpiece 10 are rotatably secured to the housing 36 by an axle 53 at laterally opposite sides of the housing 36. The rollers 51, 52 are configured to roll against the surface 11 as the housing 36 is moved longitudinally along the surface 11. When the handpiece 10 is pressed against and moved along the surface 11, the rollers 51, 52 eliminate shear forces and scraping of the surface, which might occur in the absence of rollers. The rollers 51, 52 also keep the laser optics optimally, precisely and consistently positioned above the skin for optimal and uniform focusing of the laser light as the handpiece 10 is moved along the surface 11. In this example, the handpiece 10 has two rollers 51, 52 at laterally opposite sides of the handpiece 10 to keep both sides of the handpiece 10 uniformly spaced from the surface 11, which reduces lateral tilting of the handpiece 10. A third roller (not shown), longitudinally offset from the other two rollers 51, 52, may be added to avoid longitudinal tilting of the handpiece 10. The rollers 51, 52 in this example are made of plastic, and are transparent to avoid blocking the user's view of the treatment area.

A movement sensor 54 in this example senses (e.g., measures) a linear movement parameter, which comprises longitudinal speed or longitudinal displacement (where “or” includes the possibility of both), of the housing 36 relative to the surface 11. The sensor 54 senses the handpiece's longitudinal movement indirectly, by measuring movement of a movable element that moves synchronously (in sync) with the handpiece's longitudinal movement. In this example, the moveable element comprises one of the support rollers—roller 51—that serves as a tracking roller (tracking wheel). A rate of rotation of the tracking roller 51 is proportional to speed of the handpiece's longitudinal movement. The sensor 54 senses an angular movement parameter, comprising the roller's angular speed or angular displacement, and outputs a signal that indicates an angular movement parameter (angular speed or displacement) which is also indicative of the linear movement parameter (longitudinal speed or displacement). The sensing may be based on a Hall effect sensor that detects a magnet attached to the tracking roller 51. Or the sensing may be based on an optical reader that detects interruptions in a light beam that is transmitted through a grating in the tracking roller 51 (as do some mouse mechanisms), or interruptions in a light beam that is reflected off of reflectors that are circumferentially spaced apart about the tracking roller 51. Or the sensing may be based on a mechanical electrical switch whose activation lever is pressed by protrusions that are circumferentially spaced apart about the tracking roller 51. In these examples, having at least two magnets or at least two light beams or at least two switches enables the sensor 54 to also sense direction (i.e., in addition to speed and displacement).

In one example, the rollers 51, 52 are coupled together with a common axle 53 that forces the rollers 51, 52 to rotation in unison. In that case, the sensing device 54 might monitor movement of only one of the rollers 51.

In another example, the rollers 51, 52 rotate independently of each other. In that case, the sensor 54 may sense the angular movement parameter (i.e., angular speed or displacement) of each roller 51, 52 independently, to yield two independent angular movement parameters. The sensor 54 may then sense (measure), based on the imbalance in rotation of the two rollers 51, 52, the curvature of the path of movement along the surface 11. This is especially useful where the handpiece 10 is moved along the surface 11 in an arcuate (curved) path of small radius, such that the roller at the outside of the arc moves significantly faster than the roller at the inside of the arc.

A controller 60 in the housing 36 receives the output signal of the movement sensor 54 and uses the output signal to time the pulses to be longitudinally spaced apart in a controlled manner. The longitudinal hole spacing (longitudinal distance between holes) can be reproducible and uniform despite variability in the handpiece's speed. That is because the controller 60 controls the repetition rate of the pulses 42, and thus the longitudinal spacing between holes 13, to be a function of the sensed angular movement parameter (angular speed or displacement), which is in turn a function of the linear movement parameter (longitudinal speed or displacement). For example, the controller 60 may space apart the pulses as a function of the angular displacement. The repetition rate might be proportional to the longitudinal speed if the longitudinal spacing is to be uniform. The controller 60 may cause the pulses to cease when the handpiece 10 stops moving, and automatically restart when the handpiece 10 starts moving again.

In an example that does not employ a movement sensor to adjust the repetition rate to correspond to longitudinal speed, the user moves of the handpiece 10, while the pulses are being emitted, at a speed that corresponds to pulse rate and longitudinal hole spacing in a single exposure to produce a uniform pattern. Even in such a scenario where a movement sensor is not used, the holes in each laterally-extending row may be produced simultaneously or sequentially (in a scanned manner).

If the sensor 54 senses direction (as described above), the controller 60 may detect when roller rotation has reversed direction, which indicates the handpiece 10 has reversed longitudinal direction and is returning to an area that was already ablated. In that scenario, the controller 60 would control the pulse emitter 40 to cease emitting pulses, which is equivalent to dropping the repetition rate to zero, so as not to ablate the same area twice.

If the sensor 54 sense curvature (such as by using the two-roller configuration described above), the controller 60 may control the repetition rate as a function of path curvature. For example, the controller 60 may detect that one side of the handpiece 10 is moving slower than the laterally opposite side of the handpiece 10. In such a scenario, the controller 60 might control the emitter 40 for the repetition rate to increase near the outside of the arc and/or decrease near the inside of the arc, so as to maintain a uniform hole density (i.e., keep the hole density the same at the inside of the arc as at the outside of the arc). Alternatively, or in addition, the controller 60 might control the emitter 40 for the lateral hole spacing to increase along the inside of the curve and decrease along the outside of the curve. The controller 60 might also use the curvature sensing to keep track of the handpiece's path. That would enable the controller 60 to detect when the handpiece's path of movement has curved around and is crossing over itself. The controller 60 might respond by ceasing laser pulses (i.e., lowering the repetition rate to zero) so as not to ablate the same area twice.

The controller 60 may be a mechanical device, or based on hardwired electronic logic such with an ASIC (application specific integrated circuit), or based on a microprocessor that executes program instructions stored in a memory 61 of the handpiece 10 to perform its functions. Besides storing program code, the memory 61 might be used to store sensed information regarding prior use of the handpiece, such as data that indicates the path that has been covered by the handpiece 10, to avoid ablating the same area twice.

In the procedure described above, the laterally extending rows 12 of holes 13 are spaced longitudinally apart. This results in an array (matrix) of holes 13 that are arranged in laterally extending rows 12 and longitudinally extending columns. The repetition rate of the laser pulses, and thus the row spacing (spacing between rows), is a function of the angular movement parameter (angular speed or displacement) of at least one of the rollers 51, which is in turn a function of the linear movement parameter (longitudinal speed or displacement).

During a single stroke of the handpiece 10 along the surface 11, the handpiece 10 may generate from two holes to an array of tens of thousands of holes or more. The array is a one-dimensional or two-dimensional array of ablation holes. By limiting the ablation to an array of small holes 13, the handpiece 10 might treat only 15-20 percent of the treatment area. The laser pulses 42 create, at each hole 13, an ablative thermal channel, creating a micro-injury, without disturbing the surrounding tissue. The micro-injured areas start the healing process and the surrounding untreated area 14 acts as a reservoir for rapid restoration. As collagen remodels, skin tightens, and texture and scars improve. The intact, undamaged skin around the treatment site promotes quicker healing for faster recovery.

The handpiece 10 has a user input device 62, such as a keypad or application-specific buttons or touch screen, that enables the user to set treatment parameters. The user selectable parameters may include number of holes per row, lateral spacing and longitudinal spacing between holes, hole size, and laser power intensity and pulse duration which affect ablation depth. The user may specify whether the column spacing and/or row spacing should be uniform and nonuniform, and (if nonuniform) specify what nonuniform spacing pattern to use. For example, the user may enter a selection in the input device 62 for the longitudinal spacing between holes to be non-uniform (changing), while keeping the lateral spacing within each row uniform and, if desired, keeping the lateral spacing within each row constant even from row to row.

Examples of treatment parameters are as follows: The number of pulsed beams, and thus the holes 13, in each row might be in the range 1-20, or more. The laser repetition rate might vary from 10 Hz to 15 Hz as handpiece 10 speed varies from 3.3 mm/sec to 10 mm/sec. The hole size might be in the range 150-250 um. The lateral spacing and longitudinal spacing between holes might be in the range 500-1000 um. The lateral spacing might be equal to, or not equal to, the longitudinal spacing. The laser energy in each hole may be in the range 5-20 mJ. In one example, the array of pixels may include ten columns with a column spacing (spacing between columns) in the range 600-1000 um. The handpiece's longitudinal speed along the surface 11 might be 66 mm/sec. An example hole diameter is 200 um. An example hole depth is 1 mm. There is no limit to the number of rows 12 of holes 13, since they are continually generated as the handpiece 10 is moving.

The fractional handpiece 10 described above is well suited for dermatological treatments of the face, chest, neck and hands. It is particularly well suited for reducing mild to medium wrinkling It enables quick treatment time, with no requirement for anesthesia, gels or other disposables, and with little or no downtime, and reduces risk of complications.

The components and procedures described above provide examples of elements recited in the claims. They also provide examples of how a person of ordinary skill in the art can make and use the claimed invention. They are described here to provide enablement and best mode without imposing limitations that are not recited in the claims. In some instances in the above description, a term is followed by a substantially equivalent term enclosed in parentheses.

1. A medical device comprising:
a housing configured to be moved along a surface of a target tissue in a longitudinal direction;
one or more supply lines configured to conduct air and water to the housing;
a pulse emitter configured to emit electromagnetic pulses toward the surface at a repetition rate for the pulses to produce ablation holes in the tissue, wherein the pulse emitter includes optical components and is configured to direct the air against the optical components to keep the optical components clean; and
one or more nozzles configured to emit the air and the water in an air/water spray to moisturize and cool the target tissue prior to laser application.

2. The medical device of claim 1, wherein the electromagnetic pulses are in the range 1.9-3.0 micrometers.

3. The medical device of claim 1, wherein the pulses generate laterally-extending rows of the holes, and wherein the medical device further includes:
a user input device configured for a user to enter a selection for longitudinal spacing between the holes to be non-uniform while keeping lateral spacing within each row uniform.

4. The medical device of claim 1, wherein the pulse emitter is configured to generate the holes in each row simultaneously.

5. A method performed by a user of the medical device of claim 1, comprising:
moving the housing in the longitudinal direction at a speed that is based on the pulse rate and the longitudinal hole spacing.

6. A medical device comprising:
a housing configured to be moved along a surface of a target tissue in a longitudinal direction;
a movement sensor configured to measure a movement parameter, comprising longitudinal speed or longitudinal displacement, of the housing relative to the surface;
a pulse emitter configured to emit electromagnetic pulses toward the surface at a repetition rate, for the pulses to produce ablation holes in the tissue; and
a controller configured to control the repetition rate, based on the measured movement parameter, so that the ablation holes are spaced apart along the longitudinal direction.

7. The medical device of claim 6 wherein the pulses are laser pulses.

8. The medical device of claim 6, wherein the movement parameter comprises longitudinal speed, and the sensor is configured to output a speed signal indicative of the longitudinal speed, and wherein the controller is configured to control the repetition rate to be a function of the longitudinal speed indicated by the speed signal.

9. The medical device of claim 6, wherein the movement parameter comprises longitudinal displacement, and the sensor is configured to output a displacement signal indicative of the longitudinal displacement, and wherein the controller is configured to control emission of the pulses to be a function of the displacement signal.

10. The medical device of claim 6, further comprising a roller that is rotatably secured to the housing and configured to roll against the surface as the housing is moved longitudinally along the surface, so that rate of rotation of the roller is proportional to longitudinal speed, and wherein the movement sensor is configured measure an angular movement parameter, comprising angular speed or angular displacement, of the roller.

11. The medical device of claim 10, wherein the angular movement parameter is angular speed of the roller, and the controller is configured to control the repetition rate to be a function of the angular speed.

12. The medical device of claim 10, wherein the angular movement parameter is angular displacement of the of the roller, and the controller is configured to control the emitter to space apart the pulses as a function of the angular displacement.

13. The medical device of claim 10, wherein the roller is a first roller at a first side of the housing, the angular movement parameter is a first angular movement parameter, the movement sensor includes a second roller at a laterally opposite second side of the housing, the second roller is rotatably secured to the housing to rotate independently of rotation of the first roller, and the second roller is configured to roll against the surface, and the movement sensor is configured measure a second angular movement parameter, comprising angular speed or angular displacement, of the second roller.

14. The medical device of claim 13, wherein the controller is configured to control the repetition rate as a function of both the first movement parameter and the second angular movement parameter.

15. The medical device of claim 13, wherein the pulses emitted by the emitter generate laterally-extending rows of ablation holes, and wherein the controller is configured to control the number of ablation holes in each row as a function of both the first angular movement parameter and the second angular movement parameter.

16. The medical device of claim 10, wherein the controller is configured to drop the repetition rate to zero in response to the roller reversing direction.

17. The medical device of claim 10, wherein the roller is transparent.

18. The medical device of claim 6, wherein the pulses include pulses that are laterally spaced apart to produce laterally-extending rows of ablation holes that, when combined with the longitudinal movement of the housing, yield a matrix of the holes aligned in laterally extending rows and longitudinally extending columns.

19. The medical device of claim 18, wherein the pulse emitter is configured to generate the holes in each row simultaneously.

20. The medical device of claim 18, wherein the emitter includes a diffractive optical element configured to split electromagnetic energy into a laterally extending row of simultaneous electromagnetic pulses that generate the holes simultaneously.

21. The medical device of claim 18, wherein the pulse emitter is configured to generate the holes in each row sequentially.

22. The medical device of claim 18, wherein the controller is configured for the ablation holes to be uniformly spaced apart along the longitudinal direction.

23. The medical device of claim 18, wherein the controller is configured for the ablation holes to be nonuniformly spaced apart along the longitudinal direction.

24. The medical device of claim 18, wherein the controller is configured for the ablation holes to be uniformly spaced apart along the lateral direction.

25. The medical device of claim 18, wherein the controller is configured for the ablation holes to be nonuniformly spaced apart along the lateral direction.
